(19) United States
(12) Patent Application Publication
(10) Pub. No.: US 2021/0000157 A1
(43) Pub. Date: Jan. 7, 2021

(54) EXTRUDATE COMPRISING VITAMIN A

(71) Applicant: DSM IP ASSETS B.V., Heerlen (NL)

(72) Inventors: Jihane ACHKAR, Kaiseraugst (CH); Andrea BULBARELLO, Kaiseraugst (CH); Alan CONNOLLY, Kaiseraugst (CH); Stefanie KIRCHEN, Kaiseraugst (CH); Henry RIEGER, Kaiseraugst (CH); Simon SPREITER, Kaiseraugst (CH); Kai URBAN, Kaiseraugst (CH)

(21) Appl. No.: 16/977,302

(22) PCT Filed: Mar. 14, 2019
(86) PCT No.: PCT/EP2019/056448

§ 371 Date: Sep. 1, 2020

(30) Foreign Application Priority Data
Mar. 15, 2018 (EP) 18162011.3

(51) Int. Cl. A23P 30/20; A23L 29/219; A23L 29/30; A23L 33/155; A23P 10/25

(52) U.S. Cl. CPC A23P 30/20; A23L 29/219; A23P 10/25; A23L 33/155; A23L 29/35

(57) ABSTRACT

The present invention relates to extrudates comprising a matrix and vitamin A palmitate. The matrix comprises or consists of octenyl succinate starch and dextrin. Such water-soluble or water-dispersible extrudates have an excellent storage stability and may comprise further components such as other fat-soluble vitamins.

### TECHNICAL FIELD

The present invention relates to the stability of oral dosage forms comprising vitamin A.

### BACKGROUND OF THE INVENTION

Oral dosage forms comprising vitamin A and other fat-soluble vitamins may be liquids, tablets, capsules, powders or extrudates.

Shelf-life is an important feature of any vitamin supplement. A product which has a shelf-life of less than 6 months has in many cases no commercial value.

Vitamin A is sensitive to oxygen. Therefore, vitamin A extrudates often require extensive packaging.

Vitamin powders are being sold in bags or stick packs. Such packaging systems are also suitable for extrudates.

Bags and stick packs often contain a single dose. After consumption, the empty bag is thrown away. This leads to considerable waste, particularly if such containers contain layers of aluminum foil.

There is a need for an oral dosage form having all or at least some of the following features:
contains a source of vitamin A
contains other fat-soluble vitamins (in addition to vitamin A)
has good storage stability/long shelf-life
does not require expensive, non-sustainable packaging material
has low production cost

### SUMMARY OF THE INVENTION

To reduce cost of goods, extrudates instead of pellets, tablets, capsules etc. are produced. Extrudates have reduced cost of good as they can be manufactured in a continuous manner.

Cost of goods are further reduced by providing a concentrated extrudate with a relatively small volume. Such extrudates need less packaging. This allows to reduce cost and waste.

Cost for packaging can be further reduced by providing extrudates with a high per se stability.

Extrudates comprising vitamin A are more stable if vitamin A palmitate is used as source of vitamin A. Surprisingly, stability can be further improved if vitamin A palmitate is embedded in a matrix that consists essentially of octenyl succinate starch and dextrin.

Without wishing to be bound by theory, it has been hypothesized that vitamin A palmitate is less susceptible to crystallization than vitamin A acetate within a matrix consisting essentially of octenyl succinate starch and dextrin. There are certain hints that vitamin A palmitate binds to the OH groups of dextrin which helps prevent crystallization.

Stability of extrudates comprising vitamin A can be further improved by the addition of a mixture comprising α-tocopherol, β-tocopherol, γ-tocopherol and δ-tocopherol.

In a preferred embodiment, the extrudate of the invention comprises
1-2 weight-% vitamin A palmitate, based on the total weight of the extrudate and not including any residual water,
0.001-0.02 weight-% vitamin D3, based on the total weight of the extrudate and not including any residual water,
8-15 weight-% dl-α-tocopheryl acetate, based on the total weight of the extrudate and not including any residual water,
at least 30 weight-% octenyl succinate starch, based on the total weight of the extrudate and not including any residual water,
at least 30 weight-% dextrin, based on the total weight of the extrudate and not including any residual water, and
preferably at least one antioxidant,

wherein the weight ratio between octenyl succinate starch and dextrin is from 2:1 to 1:2 and is preferably 1:1.

The method for manufacturing such extrudates comprises the steps:
feeding a mixture of octenyl succinate starch and dextrin into a first barrel of an extruder
injecting water into a second barrel being located downstream of said first barrel
injecting fat-soluble vitamins into a third barrel being located downstream of said first barrel and said second barrel.

### DETAILED DESCRIPTION OF THE INVENTION

The present invention relates to extrudates.

In the context of the present invention, the term “extrudate” refers to solid particles which are preferably water-soluble or water-dispersible. A “water-soluble” or “water-dispersible” extrudate falls apart when put into 2 dl water at a temperature of 30° C. under stirring with a spoon at 60 rpm (revolutions per minute) for less than two minutes. In a preferred embodiment of the invention, “water-soluble” and “water-dispersible” means that the extrudate falls apart when put into 2 dl water at a temperature of 22° C. under stirring with a spoon at 60 rpm (revolutions per minute) for less than two minutes

The extrudate of the invention has preferably a length from 50 μm to 2000 μm, wherein “length” is referring of the longest linear distance that can be measured. This definition of length takes into consideration that the particle might have an irregularly shape such as the shape of a potato. In case of spherical extrudates, the sphere's diameter corresponds to the length of the particle. Spherical extrudates are obtainable e.g. by spheronization of cylindrical extrudates. In a preferred embodiment of the invention, extrudates fulfil the specification 1000 μm>size>212 μm. Whether or not said specification is met is measured by sieving.

Multiple sources of vitamin A such as vitamin A acetate and vitamin A palmitate are known. Surprisingly, vitamin A palmitate has proven to be more suitable for preparing extrudates than vitamin A acetate.

Thus, the present invention also relates to the use of vitamin A palmitate to manufacture extrudates, wherein said extrudates comprise preferably a matrix as herein described.

Surprisingly, “recovery” and “stability” is improved when vitamin A palmitate is used as a source of vitamin A.

In the context of the present invention, “recovery of vitamin A” is the vitamin A content being measured by HPLC within 12 hours after extrusion and being indicated in percentages of the calculated (i.e. theoretical) vitamin A content. The recovery of vitamin A from extrudates according to the invention is preferably more than 80%, more preferably more than 90% and most preferably more than 95% of the calculated vitamin A content.

In the context of the present invention, “stability of vitamin A” refers to the vitamin A content being measured by HPLC 4 weeks after extrusion or 12 weeks after extrusion. Stability is indicated in percentages of the vitamin A content which has been measured within 12 hours after extrusion by HPLC.

Matrix

The matrix of the invention's extrudate may consist of one compound only or may comprise more than one compound. Surprisingly, recovery of vitamin A palmitate is particularly good if the matrix of the invention's extrudate consists essentially of a mixture of octenyl succinate starch and dextrin.

In the context of the present application, any compound which is present in the extrudate in an amount of at least 10 weight-% of the total weight of the extrudate (not including any residual water) is—by definition of the present patent application—part of the extrudate's matrix. Thus, the term “matrix” refers to those compounds of the extrudate which are present in an amount of at least 10 weight-% of the total weight of the extrudate (not including any residual water).

Therefore, by way of example, the matrix of an extrudate which comprises
5 weight-% X
70 weight-% Y and
25 weight-% Z

consists of compounds Y and Z.

If only one compound of the extrudate is present in an amount of at least 10 weight-% of the total weight of the extrudate (not including any residual water), the term “matrix” refers to this compound. Thus, the matrix of an extrudate which comprises
5 weight-% X
90 weight-% Y and
5 weight-% Z

consists of compound Y.

In the context of the present invention, vitamins such as vitamin A, vitamin D and α-tocopheryl acetate (being the preferred source of vitamin E) are not part of the extrudate's matrix, even if they are present in an amount of at least 10 weight-% of the total weight of the extrudate (not including any residual water). Vitamins are actives which are embedded in the extrudate's matrix.

In the context of the present invention and unless otherwise indicated, “weight-%” always refers to the total weight of the extrudate, not including any residual water (i.e. based on the dry weight of the extrudate).

In a preferred embodiment of the invention, the extrudate comprises at least 10 weight-% of an emulsifier. Thus, in such embodiment, the matrix of the extrudate comprises an emulsifier. The preferred emulsifier is octenyl succinate starch such as commercially available HiCap®.

In the context of the present invention, the term “dextrin” refers to a mixture of carbohydrates obtainable by the hydrolysis of starch or glycogen. In the context of the present invention and despite of being a mixture, “dextrin” is treated as a single compound when calculating its amount in weight-% of the total weight of the extrudate.

In a preferred embodiment of the invention, the extrudate comprises at least 10 weight-% dextrin. Thus, in such embodiment, the matrix of the extrudate comprises dextrin. Different kinds of dextrin are known and commercially available. A commercially available brand is Crystal Tex®.

Without wishing to be bound by theory, it is believed that vitamin A palmitate binds to the OH groups of dextrin which helps prevent crystallization.

Matrices comprising compounds other than octenyl succinate starch and dextrin have been tested. Surprisingly, a matrix comprising gum acacia in addition to octenyl succinate starch and dextrin does not perform as good as a binary matrix consisting of octenyl succinate starch and dextrin. Surprisingly, the same applies if the matrix comprises semolina in addition to octenyl succinate starch and dextrin. An extrudate comprising such matrix has significant more surface oil than an extrudate whose matrix consists of octenyl succinate starch and dextrin.

Thus, the matrix of the invention's extrudate preferably consists of octenyl succinate starch and dextrin. In such extrudate, the only compounds (apart from vitamins) being present in an amount of at least 10 weight-% of the total weight of the extrudate (not including any residual water) are octenyl succinate starch and dextrin.

Therefore, a preferred embodiment of the invention relates to an extrudate which comprises a matrix and vitamin A palmitate, wherein said matrix consists of octenyl succinate starch and dextrin.

In a preferred embodiment of the invention, the weight ratio between octenyl succinate starch and dextrin is from 2:1 to 1:2. Particularly preferred is a weight ratio from 1.5:1 to 1:1.5. The most preferred weight ratio is 1:1.

Therefore, a preferred embodiment of the invention relates to an extrudate which comprises a matrix and vitamin A palmitate, wherein said matrix consists octenyl succinate starch and dextrin, and wherein the weight ratio between said octenyl succinate starch and said dextrin is from 2:1 to 1:2 and is preferably from 1.5:1 to 1:1.5.

Typically, the extrudate of the invention comprises at least 30 weight-% octenyl succinate starch and preferably at least 30 weight-% dextrin, wherein the above-mentioned weight ratios between octenyl succinate starch and dextrin apply.

Therefore, a preferred embodiment of the invention relates to an extrudate which comprises a matrix and vitamin A palmitate, wherein the extrudate comprises at least 30 weight-% octenyl succinate starch and at least 30 weight-% dextrin, and wherein the weight ratio between said octenyl succinate starch and said dextrin is from 2:1 to 1:2, is preferably from 1.5:1 to 1:1.5 and is most preferably 1:1.

The person skilled in art knows how to apply this teaching. He knows, for example, that all weight percentages must add up to 100 weight-% (not taking into account any residual water, unless indicated otherwise). Thus, he would refrain from choosing weight percentages and/or weight ratios in an unreasonable manner.

Antioxidant

The extrudate of the present invention may comprise at least one antioxidant. Preferably, antioxidants are present in an amount of less than 10 weight-% of the total weight of the extrudate (not including any residual water). Thus, according to the definition of the present invention, the antioxidant is typically not part of the extrudate's matrix.

The extrudate of the invention may comprise fat-soluble antioxidants.

Thus, in one embodiment of the present invention, the extrudate comprises
a source of vitamin A such as vitamin A palmitate
at least one antioxidant and
a matrix,
wherein said matrix consist of octenyl succinate starch and dextrin, and wherein the weight ratio between said octenyl succinate starch and said dextrin is from 2:1 to 1:2.

Preferably, the extrudate comprises from 0.01 weight-% to 5 weight-% of one or more fat-soluble antioxidants based on the total weight of the extrudate (not including any residual water). Even more preferred are extrudates comprising from 0.05 weight-% to 3 weight-% of one or more fat-soluble antioxidants based on the total weight of the extrudate (not including any residual water).

Preferred fat-soluble antioxidants are α-tocopherol, β-tocopherol, γ-tocopherol and δ-tocopherol. Particularly preferred is a mixture comprising α-tocopherol, β-tocopherol, γ-tocopherol and δ-tocopherol. Such a mixture is referred to as “mixed tocopherols” and is commercially available at DSM® Nutritional Products under the brand “Mixed Tocopherols 95”.

“Mixed tocopherols 95” as available at DSM® Nutritional Products comprises α-tocopherol, β-tocopherol, γ-tocopherol and δ-tocopherol. Said tocopherols are typically (R,R,R)-tocopherols. In contrast, all-rac tocopherol is noted as dl-tocopherol.

The total tocopherol content of “mixed tocopherols 95” is at least 95 weight-%, based on the total weight of the product. It comprises more δ-tocopherol than α-tocopherol, i.e. the weight ratio α-tocopherol:δ-tocopherol in “mixed tocopherols 95” is less than 1. It also comprises more γ-tocopherol than α-tocopherol, i.e. the weight ratio α-tocopherol:γ-tocopherol in “mixed tocopherols 95” is less than 1. The weight ratio α-tocopherol:non-α-tocopherol in “mixed tocopherols 95” is less than 1, wherein the term “non-α-tocopherol” is referring to the accumulated weight of β-tocopherol, γ-tocopherol and δ-tocopherol.

Surprisingly, the extrudate of the invention is particularly stable if a mixture comprising α-tocopherol, β-tocopherol, γ-tocopherol and/or δ-tocopherol is added.

Thus, in one embodiment of the present invention, the extrudate comprises
a source of vitamin A such as vitamin A palmitate
at least one antioxidant and
a matrix,
wherein said matrix consists of octenyl succinate starch and dextrin, and wherein the weight ratio between said octenyl succinate starch and said dextrin is from 2:1 to 1:2; and
wherein extrudate comprises α-tocopherol, β-tocopherol, γ-tocopherol and/or δ-tocopherol and wherein the extrudate comprises preferably α-tocopherol, β-tocopherol, γ-tocopherol and δ-tocopherol.

In a preferred embodiment of the invention, the weight ratio between α-tocopherol and δ-tocopherol ratio is from 0.5:1 to 2:1, more preferably from 0.5:1 to 1:1 and most preferably from 0.5:1 to 0.9:1. In an also preferred embodiment of the invention, the weight ratio between α-tocopherol and γ-tocopherol ratio is from 0.5:1 to 2:1, more preferably from 0.5:1 to 1:1 and most preferably from 0.5:1 to 0.9:1.

The present invention also relates to the use a mixture comprising α-tocopherol, β-tocopherol, γ-tocopherol and δ-tocopherol for manufacturing an extrudate comprising a source of vitamin A such as vitamin A palmitate.

Fat-Soluble Vitamins

In a preferred embodiment of the invention, the extrudate comprises more than one fat-soluble vitamin. Other fat-soluble vitamins that can be added are—for example—vitamin D and vitamin E. A preferred source of vitamin E is α-tocopheryl acetate such as dl-α-tocopheryl acetate. A preferred source of vitamin D is vitamin D3.

Thus, one embodiment of the invention relates to an extrudate comprising
vitamin A palmitate,
vitamin D3,
a source of vitamin E such as α-tocopheryl acetate,
at least one fat-soluble antioxidant, and
a matrix,
wherein said matrix consists of octenyl succinate starch and dextrin, and wherein the weight ratio between octenyl succinate starch and dextrin is preferably from 2:1 to 1:2, and
wherein said at least one fat-soluble antioxidant is a mixture comprising α-tocopherol, β-tocopherol, γ-tocopherol and δ-tocopherol.

An even more preferred embodiment of the invention relates to an extrudate comprising
vitamin A palmitate,
vitamin D3,
at least 5 weight-% of a source of vitamin E such as α-tocopheryl acetate, based on the total weight of the extrudate (not including any residual water),
at least one antioxidant,
40-45 weight-% octenyl succinate starch, based on the total weight of the extrudate (not including any residual water) and
40-45 weight-% dextrin, based on the total weight of the extrudate (not including any residual water).

The most preferred embodiment of the invention relates to an extrudate comprising
vitamin A palmitate,
vitamin D3,
at least 5 weight-% α-tocopheryl acetate, based on the total weight of the extrudate (not including any residual water),
40-45 weight-% octenyl succinate starch, based on the total weight of the extrudate (not including any residual water),
40-45 weight-% dextrin, based on the total weight of the extrudate (not including any residual water), and
a mixture comprising α-tocopherol, β-tocopherol, γ-tocopherol and δ-tocopherol.

Residual Water

Typically, the extrudate of the invention is obtained by extruding a wet mixture. Therefore, the strand leaving the extruder contains a certain amount of water. Said strand is then cut into pieces. These pieces also contain water and may need drying. Drying can be more or less thorough. Thus, the extrudate of the invention may or may not comprise residual water. The term “residual water” refers to an amount of water not exceeding 10 weight-% of the total weight of the extrudate, including said residual water. Typical residual water levels are between 4-6 weight-% of the total weight of the extrudate, including said residual water. Excessive drying is to be avoided as it can lead to loss of vitamins due to heat and oxidation.

Manufacturing Method

The present invention also relates to a method for manufacturing extrudates as herein described.

In one embodiment of the invention, said method comprises the steps:
feeding a mixture of octenyl succinate starch and dextrin into a first barrel of an extruder
injecting water into a second barrel being located downstream of said first barrel
injecting at least one fat-soluble vitamin into a third barrel being located downstream of said first barrel and said second barrel.

Typically, an extruder is used that has more the three barrels. Thus, said first barrel may or may not be separated for said second barrel by one or multiple barrels. Similarly, said second barrel may or may not be separated from said third barrel by one or multiple barrels.

Another embodiment of the invention relates to a method for manufacturing an extrudate comprising a source of vitamin A, wherein said method comprises the steps:
feeding a pulverulent mixture into a first barrel of an extruder
injecting water into a second barrel being located downstream of said first barrel, and
injecting a composition comprising vitamin A palmitate into a third barrel being located downstream of said first barrel and said second barrel.

In a preferred embodiment of the invention, said method comprises the steps:
feeding a mixture of octenyl succinate starch and dextrin into a first barrel of an extruder
injecting water into a second barrel being located downstream of said first barrel, and
injecting a composition comprising vitamin A palmitate into a third barrel being located downstream of said first barrel and said second barrel.

The extruder to be used in the method of the invention has at least 3 barrels, preferably at least 4 barrels and most preferably at least 6 barrels. Preferably, the third barrel is separated from the second barrel by at least one barrel. Thus, in a preferred embodiment of the invention, dry pulverulent matrix material is fed into barrel 1, distilled water is fed into barrel 2 and the fat-soluble vitamin(s) is/are fed into barrel 4.

Preferably, the extruder to be used in the method of the invention has a l/d ratio from 15 to 40, preferably from 20 to 30 and most preferably from 22 to 26, wherein “l” means screw length and wherein “d” means screw diameter.

In one embodiment, the extrudate of the invention comprises multiple fat-soluble vitamins. Depending on the melting point of the mixture, it is preferred to melt the mixture before injecting the mixture into the extruder. Thus, one embodiment of the invention relates to a method for manufacturing an extrudate comprising multible fat-soluble vitamins, wherein said fat-soluble vitamins and optionally at least one fat-soluble antioxidant are molten before being injected into above-mentioned third barrel, which is located downstream of above-mentioned first barrel and above-mentioned second barrel. This method is particularly preferred for manufacturing extrudates comprising vitamin D3 as a source of vitamin D.

Another embodiment of the invention relates to a method for manufacturing an extrudate comprising a source of vitamin D3, wherein said method comprises the following the steps:
feeding a pulverulent mixture into a first barrel of an extruder
injecting water into a second barrel being located downstream of said first barrel and
injecting a composition comprising vitamin D3 and optionally vitamin A palmitate into a third barrel being located downstream of said first barrel and said second barrel,

wherein said composition comprising vitamin D3 and optionally vitamin A palmitate is molten before being injected into said third barrel being located downstream of said first barrel and said second barrel, and wherein said third barrel is preferably separated by at least one barrel from said second barrel.

Preferably, the extruder is fitted with a die having multiple holes, said holes having a diameter from 0.2 mm to 1.5 mm, preferably from 0.5 mm to 1 mm.

During the extrusion, the extruder as such is neither heated nor cooled, i.e. extrusion takes place under adiabatic conditions. The screw, screw speed, feed rate and temperature of the injected composition (if applicable) is preferably chosen such that after about 60 minutes of continuous extrusion, the temperature at the die remains approx. constant at temperature from preferably 60° C. to 95° C., more preferably from 70° C. to 90° C.

Preferably, die face cutting is done once the temperature at the die remains approx. constant in the above-mentioned ranges. The extrudates may then be dried e.g. on a fluid bed dryer, if needed or desired.

In a preferred embodiment, the obtained extrudates are then are then sieved (1000 μm>size>212 μm) to exclude particles that are too large or too small.

### Example 1 (Vitamin a Palmitate Vs. Acetate)

Storage stability of extrudates comprising vitamin A palmitate is compared with storage stability of extrudates comprising vitamin A acetate. Two different matrices were used. Both matrices consisted of octenyl succinate starch (HiCap® 100) and dextrin (weight ratio=1:1). However, different kinds of dextrin (Crystaltex® 644 and maltodextrin DE 0508, respectively) were used. Dextrins may be characterized by indicating a DE (dextrose equivalent) value. Maltodextrin DE 0508 is commercially available as Glucidex 6 (Roquette).

Extrudates were generated on a Haake Polylab drive (Thermo Fischer, Karlsruhe) unit connected to a Rheomex PTW16/25 OS Twin Screw extruder with an l/d ratio of 25 fitted with a 0.8 mm die consisting of 15 holes (Thermo Fischer, Karlsruhe).

The extruder had 6 barrels, numbered as barrel 1, barrel 2 etc. up to barrel 6. Dry pulverulent matrix material was fed into barrel 1 using a Brabender Gravimetric feeder (Thermo Fischer, Karlsruhe). Distilled water was fed by HPLC pumps with inline filters into barrel 2, said barrel 2 being located downstream of barrel 1. A molten mixture of the respective vitamin A ester (palmitate or acetate), dl-α-tocopheryl acetate (as a source of vitamin E), vitamin D3 and dl-α-tocopherol (as fat-soluble antioxidant) was fed at 80° C. into barrel 4, said barrel 4 being located downstream of barrels 1 and 2. Thermal heating had been applied to oil feed lines to ensure that temperature is maintained. Oil feed lines are not considered as being part of the extruder as such.

During extrusion, the extruder as such was neither heated nor cooled, i.e. extrusion took place under adiabatic conditions. After about 60 minutes of continuous extrusion, the temperature at the extruder's die remained stable at approximately 80° C.

Once strands appeared at the die, die face cutting began. The extrudates were then dried on a fluid bed dryer. The dried extrudates typically contained of 4-6 weight-% residual water. The extrudates were then sieved to retain and store extrudates having a particle size from 212 μm to 1000 μm.

TABLE 1
matrices used in Example 1; the respective extrudates were identical
apart from the source of vitamin A (palmitate vs. acetate)

| | matrix 1 | matrix 2 |
|---|---|---|
| vitamin A palmitate | HiCap ®100 | HiCap ®100 |
| | Maltodextrin DE 0508 | Crystal Tex ® 644 |
| vitamin A acetate | HiCap ®100 | HiCap ®100 |
| | Maltodextrin DE 0508 | Crystal Tex ® 644 |

TABLE 2
stability of vitamin A: content of vitamin A palmitate and
vitamin A acetate, respectively, being measured 4 weeks after
extrusion and being indicated in percentages of the vitamin
A palmitate/acetate content which has been measured within
12 hours after extrusion by HPLC. Extrudates were stored
in plastic tubes at 40° C. and 75% relative humidity.

| | matrix 1 | matrix 2 |
|---|---|---|
| vitamin A palmitate | 90% | 90% |
| vitamin A acetate | 60% | 63% |

TABLE 3
stability of vitamin A: content of vitamin A palmitate and vitamin
A acetate, respectively, being measured 12 weeks after extrusion
and being indicated in percentages of the vitamin A palmitate/acetate
content which has been measured within 12 hours after extrusion
by HPLC. Extrudates were stored in sealed aluminum pouches
at 30° C. and 65% relative humidity.

| | matrix 1 | matrix 2 |
|---|---|---|
| vitamin A palmitate | 90% | 90% |
| vitamin A acetate | 47% | 67% |

Example 1 clearly shows that extrudates comprising vitamin A palmitate are more stable than extrudates comprising vitamin A acetate. Example 1 also shows that different kinds of dextrin can be used.

### Example 2 (Binary Vs. Ternary Matrix)

The effect of different matrices on stability of extrudates comprising vitamin A palmitate was tested.

Two different extrudates were manufactured as described in example 1.

The matrix of extrudate #70 consisted of octenyl succinate starch and dextrin, i.e. extrudate #70 had a binary matrix.

The matrix of extrudate #83 consisted of octenyl succinate starch, dextrin and a gum acacia, i.e. extrudate #83 had a ternary matrix. According to the definition of the present invention, gum acacia is part of the matrix of extrudate #83 because extrudate #83 comprises more than 10 weight-% gum acacia, based on the total weight of the extrudate, not including any residual water.

Maltodextrin commercially available as Glucidex® 6 was used as dextrin.

TABLE 4
Storage stability of vitamin A: vitamin A palmitate content,
being measured 12 weeks after extrusion and being indicated
in percentages of the vitamin A palmitate/acetate content which
has been measured within 12 hours after extrusion by HPLC.

| | matrix | | | |
|---|---|---|---|---|
| | Weight-% | | Weight-% gum | Vitamin A |
| | dextrin, | Weight-% | acacia colloids, | palmitate |
| | based on | HiCap, based | based on the | content in % |
| Extrudate | the total | on the total | total dry | of initial |
| # | dry weight | dry weight | weight | content |
| 70 | 43.4 | 43.4 | none | 90% |
| 83 | 43.4 | 30.4 | 13.0 | 76% |

A comparison between extrudate #70 and extrudate #83 shows that very good storage stability is achieved if the matrix of the extrudate consists of octenyl succinate starch and dextrin.

### Example 3 (Surface Oil)

Surface oil of several extrudates comprising vitamin A palmitate were tested. Extrudates were manufactured as described in example 1. However, one fat-soluble vitamin only (i.e. vitamin A palmitate) was added.

Three different matrices materials were tested. The compositions of the respective matrices are shown in Table 5. According to the definition of the present invention, semolina is part of the matrix of extrudates #102 and #108 because said extrudates comprise more than 10 weight-% semolina, based on the total weight of the extrudate, not including any residual water.

Surface oil was then determined as follows: 1 g of extrudates was added to 40 mL of cyclohexane. The resulting suspension was then agitated on a Shaker for 30 min to dissolve any surface oil. The suspension was then centrifuged at 4000 rpm for 10 min and the resulting supernatant was diluted to 100 mL with ethanol. After mixing the resulting solution was then analysed by RP-HPLC.

A small amount of surface oil indicates good emulsification properties whereas a large amount of surface oil indicates poor emulsification properties. Surface oil has usually a detrimental effect for storage stability, in particular when the extrudate comprises an oxidable active such as vitamin A.

TABLE 5
surface oil, measured for extrudates having different matrices.
Weight-% are based on the total dry weight of the extrudate,
i.e. residual water has not been taken into account. Extrudates
contain typically 4-6 weight-% residual water.

| | matrix | | | |
|---|---|---|---|---|
| | Weight-% | | | |
| | Crystal | Weight-% | Weight-% | Surface oil |
| | Tex ®, | HiCap ®, | semolina, | in weight-%, |
| | based on | based on | based on | based on the |
| Extrudate | the total | the total | the total | total vitamin |
| # | dry weight | dry weight | dry weight | A content |
| 102 | 43.4 | none | 42.5 | 10.51 |
| 108 | 28.65 | 28.65 | 28.65 | 18.52 |
| 111 | 43.4 | 43.4 | none | Not |
| | | | | detectable |

Table 5 shows that extrudates having very low surface oil can be achieved if the matrix of the extrudate consists of octenyl succinate starch and dextrin.

Extrudates having low amounts of surface oil are generally more storage stable than extrudates having high amounts of surface oil.

### Example 4 (Fat-Soluble Antioxidant)

Four different kinds of extrudates comprising vitamin A palmitate were manufactured as described in the previous examples.

Said four different kinds of extrudates were identical apart from the content of a fat-soluble antioxidant.

To test the effect of antioxidants, the content of vitamin A palmitate was measured after 4 weeks storage at 40° C. and 75% relative humidity (1 extrudate per plastic tube; plastic tubes have a specified permeability to both oxygen and moisture). The result of said test is shown in Table 6.

TABLE 6
vitamin A palmitate content, being measured 4 weeks after extrusion
and being indicated in percentages of the vitamin A palmitate
which has been measured within 12 hours after extrusion by HPLC

| | Content of | Total content of | vitamin A |
|---|---|---|---|
| | antioxidant(s), | antioxidant(s), | palmitate content |
| | based on the | based on the | after 4 weeks, |
| | total weight | total weight | in percentages |
| Extrudate | of the dry | of the dry | of the initial |
| # | extrudate | extrudate | content |
| #104 | none | None | 46% |
| #108A | 0.16 weight-% | 0.16 weight-% | 62% |
| | dl-α-tocopherol | | |
| #108B | 0.16 weight-% | 0.16 weight-% | 67% |
| | “mixed | | |
| | tocopherols 95” | | |
| #108C | 0.08 weight-% | 0.16 weight-% | 61% |
| | dl-α-tocopherol | | |
| | 0.08 weight-% | | |
| | “mixed | | |
| | tocopherols 95” | | |

Example 4 shows that the addition of a fat-soluble antioxidant is beneficial, in particular when tocopherol is added.

### Example 5 (Application of Extrudate)

Extrudates were generated on a Haake Polylab drive (Thermo Fischer, Karlsruhe) unit connected to a Rheomex PTW16/25 OS Twin Screw extruder with an l/d ratio 25 fitted with a 0.8 mm die consisting of 15 holes (Thermo Fischer, Karlsruhe).

The extruder had 6 barrels, numbered as barrel 1, barrel 2 etc. up to barrel 6.

Dry pulverulent matrix material was fed into barrel 1 using a Brabender

Gravimetric feeder (Thermo Fischer, Karlsruhe). Distilled water was fed by HPLC pumps with inline filters into barrel 2, said barrel 2 being located downstream of barrel 1. A molten mixture of the respective vitamin A ester, vitamin D3 and dl-α-tocopheryl acetate was fed at 80° C. into barrel 4, said barrel 4 being located downstream of barrels 1 and 2. Thermal heating had been applied to oil feed lines to ensure temperature is maintained.

Barrel 4 was separated from barrel 2 by one barrel (i.e. separated by barrel 3).

During the extrusion, the extruder as such was neither heated nor cooled, i.e. extrusion took place under adiabatic conditions. After about 60 minutes of continuous extrusion, the temperature at the die remained stable at approximately 80° C.

Once the temperature at the die reached 80° C., die face cutting began. The extrudates were then dried on a fluid bed dryer. The dried extrudates typically contained residual water of 4-6 weight-% of the total weight of the extrudate. The extrudates were then sieved and extrudates having a particle size from 212 μm to 1000 μm were retained and stored.

TABLE 7
calculated composition of dried extrudates, based on
the total dry weight (i.e. ignoring residual water)

| | weight-%, based on the |
|---|---|
| Ingredient | total weight of the extrudate |
| octenyl succinate starch | 40-45 |
| (HiCap ® 100) | |
| Dextrin (Crystal Tex ® 644) | 40-45 |
| vitamin A palmitate | 1-2 |
| vitamin D3 | 0.01-0.05 |
| vitamin E acetate | 10-15 |
| fat-soluble antioxidants | 0.01-0.05 |

In the example shown in Table 7, the matrix of the extrudate consists of octenyl succinate starch and dextrin. Vitamin E acetate is not part of the matrix because it is an active (cf. definition of “matrix” as used in the context of the present invention). The extrudates were then sieved to retain and store extrudates having a particle size from 212 μm to 1000 μm.

One extrudate was put into 2 dl water at a temperature of approx. 22° C. and fell apart under stirring with a spoon at 60 rpm (revolutions per minute) for less than two minutes. Thus, the multivitamin extrudates of example 5 were cold water-dispersible.

1. Extrudate comprising a matrix and vitamin A palmitate, wherein said matrix comprises or consists of octenyl succinate starch and dextrin.

2. Extrudate according to claim 1, wherein the weight ratio between octenyl succinate starch and dextrin is from 2:1 to 1:2 and is preferably from 1.5:1 to 1:1.5.

3. Extrudate according to claim 1, wherein said extrudate is water-soluble or water-dispersible and has preferably a length from 50 μm to 2000 μm.

4. Extrudate according to claim 1, wherein said extrudate comprises at least one fat-soluble antioxidant.

5. Extrudate according to claim 1, wherein said extrudate comprises α-tocopherol and δ-tocopherol and preferably comprises α-tocopherol, β-tocopherol, γ-tocopherol and δ-tocopherol.

6. Extrudate according to claim 1, said extrudate comprising
1-2 weight-% vitamin A palmitate, based on the total weight of the extrudate and not including any residual water,
0.001-0.02 weight-% vitamin D3, based on the total weight of the extrudate and not including any residual water,
8-15 weight-% dl-α-tocopheryl acetate, based on the total weight of the extrudate and not including any residual water,
at least 30 weight-% octenyl succinate starch, based on the total weight of the extrudate and not including any residual water,
at least 30 weight-% dextrin, based on the total weight of the extrudate and not including any residual water, and
preferably at least one antioxidant,
wherein the weight ratio between octenyl succinate starch and dextrin is from 2:1 to 1:2 and is preferably 1:1.

7. Container, comprising multiple extrudates according to claim 1.

8. Container according to claim 7, wherein said container is a sachet, a bag or a stick pack and/or wherein each of said extrudates comprise at least 1 weight-% vitamin A palmitate, based on the total weight of the extrudate, not including any residual water.

9. Method for manufacturing an extrudate comprising at least one fat-soluble vitamin, said method comprising the steps:
feeding a mixture of octenyl succinate starch and dextrin into a first barrel of an extruder
injecting water into a second barrel being located downstream of said first barrel
injecting said at least one fat-soluble vitamins into a third barrel being located downstream of said first barrel and said second barrel.

10. Method according to claim 9, wherein the weight ratio between octenyl succinate starch and dextrin is from 2:1 to 1:2 and is preferably from 1.5:1 to 1:1.5 and is most preferably 1:1.

11. Method according to claim 9, wherein the extrudate comprises vitamin A palmitate.

12. Method according to claim 9, wherein a mixture of vitamin A palmitate, vitamin D3, dl-α-tocopheryl acetate and optionally at least one fat-soluble antioxidant is molten before being injected into said third barrel, and wherein said third barrel is preferably separated from said second barrel by at least one barrel.

13. Use of a vitamin A palmitate for manufacturing an extrudate, wherein said extrudate comprises octenyl succinate starch and dextrin.

14. Use according to claim 13, the weight ratio between octenyl succinate starch and dextrin is from 2:1 to 1:2 and is preferably from 1.5:1 to 1:1.5 and is most preferably 1:1.

15. Use according to claim 13, wherein said extrudate is water-soluble or water-dispersible and has preferably a length from 50 μm to 2000 μm.
